(19) United States
(12) Patent Application Publication
(10) Pub. No.: US 2013/0295170 A1
(43) Pub. Date: Nov. 7, 2013

(54) COMPOSITIONS FOR CONTROL OF DRUG ABUSE

(71) Applicant: KYDES PHARMACEUTICALS, Halethorpe, MD (US)

(72) Inventor: Stephen Dordunoo, Halethorpe, MD (US)

(73) Assignee: KYDES Pharmaceuticals LLC, Halethorpe, MD (US)

(21) Appl. No.: 13/886,962

(22) Filed: May 3, 2013

Related U.S. Application Data
(60) Provisional application No. 61/642,024, filed on May 3, 2012.

(51) Int. Cl. A61K 9/14; A61K 9/16; A61K 9/20

(52) U.S. Cl. CPC A61K 9/14; A61K 9/2072; A61K 9/16
USPC 424/451; 424/489; 514/282; 424/499; 424/502; 424/464

(57) ABSTRACT

Opiates, amphetamines, barbiturates and other drugs such as benzodiazepines are extensively abused or misused and are frequently the cause of death by overdosing. These drugs are also prone to oxidation and the final degradation products depend on the reactants and the reaction conditions. This invention describes the use of inactivating agents such as permanganates, peroxides, persulfates, bismuthates, periodates or other oxidants in a dosage form as an approach to minimize abuse and overdose. The product is designed such that the inactivating agent is released if there is an attempt to extract the drug from the formulation or in cases of overdose. Once released, the inactivating agent quickly degrades the drug and converts it into inactive compounds. Since the reactants (drug and inactivating agent) are incompatible in situations of normal drug usage, they are kept separated within the vehicle of the invention, but released for interaction in case of misuse. A catalyst may be included in the formulation to facilitate the reaction.

### CROSS-REFERENCE TO RELATED APPLICATIONS

This application claims priority under 35 U.S.C. §119(e) of U.S. Provisional Application 61/642,024 entitled “Compositions for Control of Drug Abuse and Overdose Minimizing Pharmaceutical Formulations of Opiates and Other Drugs of Abuse” filed on May 3, 2012, and which is incorporated herein by reference in its entirety.

### FIELD OF THE INVENTION

This invention relates to the field of pharmaceutical compositions and in particular to provision of a drug-antidote combination in a single delivery vehicle to minimize the risk and effect of drug abuse or misuse.

### BACKGROUND OF THE INVENTION

The need to treat pain with effective agents such as opiates while minimizing the risk of abuse and addiction became apparent shortly after the introduction of opiates into pharmaceutical/medical use several centuries ago. From archeological digs, the earliest use of opiates dates back over 30,000 years to the Neanderthals while recorded use of opium dates to 3500 BC by the Sumerians. Hence, the recreational use of drugs affecting the central nervous systems such as opiates and sleep disorder medications dates back to antiquity. Throughout history, opiates have wreaked havoc on the people of several nations and civilizations, and the need for risk mitigation approaches that optimize their risk-benefit ratio is as dire today as it has been since antiquity. In spite of extensive prevention, education and law enforcement efforts, chronic abusers are able to obtain and abuse controlled products.

Since the discovery of opiates and the isolation of morphine in the 1800's, there has been an ongoing concerted search for new molecular entities with little or no abuse potential that can be used in medicines to treat medical conditions. To date, no compound has been found that did not possess in some measure the addiction liabilities of morphine.

Most of the initial efforts concentrated on medicinal chemistry to synthesize new compounds that can be used to treat pain without abuse or addiction. Although the synthetic approach has to date largely failed, some compounds such as peripherally active opiates are being investigated and their usefulness in the treatment of all types of pain where centrally active opiates are known to be effective remains to be seen.

With the failure of synthetic approaches, abuse-deterrent formulations (ADFs) have emerged as a means for supporting access to opiates for pain management while limiting abuse and its consequences. Several different types of ADFs have emerged including physical barriers to tampering, agonist-antagonist formulations, aversion-inducing combinations, pro-drugs that target peripheral opiate receptors, and alternative methods of administration. While each of these types theoretically has the potential to reduce specific forms of prescription opioid abuse, they will require not only technically successful formulations, but also appropriate scientific assessment, widespread market penetration, and rational expectations of their benefits. Furthermore, they will not resolve the effects of overdose, if overdose occurs, despite efforts to control taste or targeted delivery.

In addition to the abuse and addiction potential of opiates, toxicity including death resulting from intentional and unintentional overdose of prescription opiates is also a significant public health burden whose incidence is on the increase worldwide. The net effect of the abuse and addiction potential of opiates is the severe limitation of the important drugs for genuine medical treatment resulting in patients suffering from undue pain. According to the World Health Organization (WHO) and the International Narcotics Control Board (INCB), millions of patients, especially in developing nations, suffer undue pain due to the unavailability or restrictive prescription of opiate pain medications. The development of prescription opiates which may not be abused “as is” (i.e. without tampering) and/or which will minimize overdose potential is a sine qua non to the availability of opiates for pain relief.

Recreational use of drugs affecting the central nervous systems, such as opiates and sleep disorder medications, dates back to antiquity. In spite of extensive prevention, education and law enforcement efforts, chronic abusers are able to obtain and abuse controlled products. Since the discovery of opiates and the isolation of morphine in the 1800's, there has been ongoing concerted search for new molecular entities with little or no abuse potential that can be used in medicines to treat medical conditions. To date, no compound has been found that did not possess in some measure the addiction liabilities of morphine.

In the absence of compounds without addiction potential, various formulation approaches have been evaluated in attempts to develop products containing abuse-able substances that provide greater deterrent to diversion and abuse than existing products. These products are designed to maintain effectiveness, safety and ease of use. In addition to reducing diversion and/or abuse, successful abuse deterrent products will also encourage physicians to prescribe these medicines for increased treatment of pain and other conditions. Abuse-deterrent formulations may also increase compliance.

### SUMMARY OF THE INVENTION

It is an object of the present invention to overcome the challenges of the prior art. In one embodiment, a drug formulation is provided that comprises an active component, a degrading component capable of degrading the active component, and a membrane separating the active component from the degrading component, wherein the membrane prevents degradation of the active component by the degrading component.

Another object of the present invention is to provide a drug formulation where the membrane is resistant to gastric fluids. In one embodiment, the membrane degrades after the pH reaches a predetermined pH. In one preferred embodiment, the predetermined pH is equal or greater than 5. In some embodiments, the formulation contains a pH enhancer such as an antacid, magnesium oxides, sodium bicarbonate, sodium acetate, and derivatives thereof.

In another object of the present invention, the active component of the drug formulation is an opiate, an amphetamine, a barbiturate, benzodiazepines or a digoxin. In one embodiment, the opiate is morphine, hydromorphone, levorphanol, codeine, thebaine, papaverine, or derivatives thereof. In another embodiment, the amphetamine is methamphetamine [(2S)-N-methyl-1-phenylpropan-2-amine]), MDA (3,4-Methylenedioxyamphetamine), MDMA (3,4-Methylenedioxy-N-methamphetamine), Amphetamine/Benzedrine (1-phenylpropan-2-amine), dextroamphetamine ([(2S)-1-phenylpropan-2-amine]), 4-FMP ([1-(4-fluorophenyl)propan-2-amine]), 4-MTA ([1-[4-(methylthio)phenyl]propan-2-amine], Benzphetamine ([(2S)-N-benzyl-N-methyl-1-phenylpropan-2-amine], or derivatives thereof.

In yet further embodiments, the formulation comprises a barbiturate such as allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), and phenobarbital (5- ethyl-5-phenylbarbiturate). In other embodiments, the formulation comprises a pH enhancer.

### DETAILED DESCRIPTION

The invention provides a drug delivery vehicle which contains a built-in antidote/inactivator of the drug, in addition to the drug, to prevent the negative consequences of overuse or misuse of the drug. Generally, the vehicle works well with drugs which normally are, or can be, prescribed in solid form (powders, pills, capsules and such). In a preferred embodiment, the drug vehicle of the invention is ingested orally. Almost any drug may be incorporated in the vehicle. Most advantageously, the invention is applied to drugs which are considered dangerous, such as drugs that lead to habit formation or endanger life if overused. Examples would include opiates, amphetamines, digioxin and such.

The delivery vehicle comprises at least two separate volumes and two different membranes, in which at least five components are present.

One component (Component 1) is a chemical that can increase the pH in a bodily fluid, preferably the stomach. The stomach has an acidic pH, between about pH 1 and pH 3. A normal dose of the drug delivered within the vehicle of the invention to a subject/patient would deliver the pH changing Component 1, but the amount delivered would not suffice to increase the pH very much, not sufficient to make the bodily fluid reach above pH 3 or pH 4. However, taking multiple doses of the drug within a short time frame will raise the pH above a critical point, say above pH 3; 3.1; 3.2 and so on, preferably at least or above pH 4. Examples of Component 1 include an antacid, magnesium oxides, sodium bicarbonate, and sodium acetate.

Another component (Component 2) is a catalyst which enhances the activity of Component 5, the drug's Inactivator. Examples include iron sulfates and EDTA. The third component, Component 3, is the drug. Examples would include opiates, amphetamines, benzodiazepines, digioxin and such.

Component 4 is a coating or formulation which is pH sensitive, i.e. will not dissolve at pH lower than pre-designated pH, e.g. at least pH 3, pH 4, or above. Some examples include Eudragit, ethylcellulose, or other polymers whose stability is pH sensitive.

Component 5 is the drug's Inactivator. It can be an oxidation agent, or a hydrolysis agent, or an agent that complexes the drug and precipitates the drug out of solution, and so on. Some examples include potassium permanganate (best against opiates), sodium (or other salts) of bismutate or persulfate, and peroxides. Tanic acid would be a preferred complex-formation/drug-precipitation agent. Of course, the drug and inhibitor are matched for suitability. For example, hydrogen peroxide may not inactivate oxycontin. By contrast, EDTA or ion sulfates would inactivate oxycontin.

Certain aspects of the invention are critical. The Inactivator resides in a compartment (seed, or core) whose coating is the pH sensitive component. The seed or core may comprise other components, for example the catalyst, or the catalyst might reside elsewhere in the vehicle. The pH changing (increasing) component is not present within the seed or core.

Upon the patient's taking a normal dose, the core/seed is not breached and it is eventually eliminated from the body. However, if enough drug/vehicle is ingested or otherwise internalized, the pH would increase, the pH sensitive formulation or coating (Component 4) would be breached and the drug antidote would be released.

Similarly, if a drug abuser were to try to grind or dissolve a drug residing in the vehicle, the drug and the drug antidote would no longer be separated and the drug would become neutralized.

An oxidant-catalyst core is coated with a polymer membrane to protect it from release when administered orally as recommended. The coated core will then be over-coated with the drug and the release-modulating membrane. Following oral administration, the drug is released but the oxidant core passes through the gastrointestinal tract without being released. The sequestering membrane protecting the oxidant is designed to breakdown on tampering which includes chewing, crushing, hot water extraction and non-aqueous solvent extraction to allow for the simultaneous release of the oxidant, the catalyst and the drug. Other forms of separation of the drug and the oxidant in the same dosage form to reduce abuse will be investigated.

The dosage form provides immediate and/or extended-release of the drug while the inactivator (e.g. potassium permanganate) remains sequestered within the dosage form. When swallowed whole either as a capsule or as pellets which can be sprinkled on apple sauce, the dosage form will provide immediate or extended release of the drug. However when crushed, chewed or extracted with hot water or other solvents, the inactivator is immediately released which will subsequently inactivate the drug (e.g. by oxidation), thus converting it into inactive products. This unique combination of inactivator and active drug is intended to reduce the illicit use of the drugs.

In another form, the inactivator is encapsulated in a pH-sensitive membrane such as enteric coating and subsequently blended with the drug and a pH modifying compound such as magnesium oxide or other alkanizing compound. When this dosage form is taken in the correct doses, the drug and the pH-modifier are released for absorption in the stomach while the inactivator is leased in the intestines. The effect of the drug is realized without inactivation since the amount of the inactivator is not enough to increase the gastric pH to levels that will cause the enteric coating to break-down to release the inactivator. But when more than the prescribed dose of the drug is swallowed, the amount of pH-modifier released will increase the gastric pH, causing the breakdown of the enteric coat and catalyzing the degradation of the drug in the stomach. The drug included in the dosage form may be a shorter acting opiate such as hydromorphone or longer acting such as levorphanol.

The inactivator may be potassium permanganate, sodium, potassium or ammonium bismuthate, sodium potassium or ammonium persulfate or other strong oxidants.

In a preferred embodiment, the drug product is made up of the drug and functional layers as described below:
1. Oxidant (inactivator) Core
2. Sequestering and/or pH-sensitive membrane
3. Drug layer
4. Drug release modifying membrane (for extended release formulations)
5. pH-modifying and/or catalyst layer (if needed for minimizing overdose risk).
   Initially, the inactivator such as potassium or sodium permanganate, sodium bismutate and/or sodium persulfate cores will be layered with a sequestering or pH-sensitive membrane.

Encapsulated oxidizer spheres/particles of suitable size (e.g. 0.15-1 mm) are prepared with or without binders by spray drying a concentrated solution or suspension of micronized particles of the oxidant using a concentric nozzle spray dryer (e.g. Buchi 390 Encapsulator). A solution or suspension of an appropriate polymer or film forming material suitable for sequestration is used as the encapsulating material. The sequestering or pH-modifying coat composition is such that it will allow the seed coat to break down during chewing, crushing, hot water or other solvent extraction or when an excessive amount of the drug is consumed.

For the enteric or sequestering membrane, the oxidant-catalyst core will be coated with a sequestering or pH-sensitive polymeric membrane. This is a critical parameter or attribute of this product. The purpose of the sequestering membrane is to completely block oxidant release for a prolonged period of time to prevent release of the oxidant in the body but release the oxidant immediately upon tampering. In the case of pH-sensitive membrane, the coating will break down in the small or large intestines after the drug is released in the stomach or duodenum. The sequestering or pH-sensitive membrane needs to be water insoluble and have minimal ability to swell throughout the physiological pH range. Mechanically, the membrane needs to be strong enough to withstand normal manufacturing processing but brittle enough to be easily cracked when the pellet is tampered with.

Lipids, waxes, ceramic materials, silicates, polysiloxanes and polymers can be used as sequestrating membrane components. Criteria for selection will be melting point between 40-70° C., hydrophobicity, film-forming ability, solubility or ability to swell in hot water or organic solvents, and fragility. Examples in sequestrating membrane component include polycaprolactone, tristearin, cetyl palmitate, cetyl alcohol and waxes. The plasticizer will be selected from dibutyl sebacate and glyceryl monostearate which are also water-insoluble. Talc or other suitable glidant may be included in the components of the sequestrating membrane.

The pH-modifier can be selected from antacids such as magnesium oxide, magnesium hydroxide, calcium hydroxide, calcium carbonate, sodium carbonate, sodium bicarbonate and other antacids.

The coated oxidizer may be over-coated with the antacid layer or blended with the antacid and compressed into tablets or filled into capsules for administration. The antacid is present in amounts such that when the recommended dose is administered, the gastric pH will not increase to a level that will cause the breakdown of the sequestrating layer to release the oxidizer. However, when administered in excess, the amount of the antacid will be enough to break down the enteric coating, causing the degradation of the drug. An additional benefit of a magnesium-based antacid may be for the management of conditions such as opioid-induced constipation.

The compositions herein described may be immediate release or controlled release dosage forms.

| | amount | Comp | % of Total |
|---|---|---|---|
| Ingredient | (mg) | (%) | composition |
| Magnesium oxide | 200 | 62.3 | 43.2 |
| Sodium acetate | 100 | 31.2 | 21.6 |
| EDTA | 1 | 0.3 | 0.2 |
| Hydroxypropyl | 20 | 6.2 | 4.3 |
| methylcellulose (HPMC) | | | |
| Sub-Total | 321 | 100.0 | 69.3 |

| Drug composition | amount | Comp | % of Total |
|---|---|---|---|
| component | (mg) | (%) | composition |
| Morphine | 20 | 74.1 | 4.3 |
| Talc | 2 | 7.4 | 0.4 |
| Hydroxypropyl | 4 | 14.8 | 0.9 |
| methylcellulose (HPMC) | | | |
| or appropriate | | | |
| Other excipients | 1 | 3.7 | 0.2 |
| Sub-total | 27 | 100.0 | 5.8 |

| | amount | Comp | % of Total |
|---|---|---|---|
| Inactivator component | (mg) | (%) | composition |
| Sodium persulfate | 75 | 65.2 | 16.2 |
| Sodium or Potassium | 25 | 21.7 | 5.4 |
| permanganate | | | |
| Eudragit, Silicate, | 10 | 8.7 | 2.2 |
| polysiloxanes or appropriate | | | |
| GMS and/or other | 5 | 4.3 | 1.1 |
| excipients | | | |
| Sub-total | 115 | 100.0 | 24.8 |
| Dosage form Total | 463 | | 100.0 |

Additional layers or film may be applied to improve the stability and/or release of the drug as needed. The final beads/pellets/particles may be used in a suspension, tablet or capsule form for administration. For suspension formulations, an appropriate final coating (e.g. Eugradit E for suspensions in neutral or slightly alkaline vehicle) may be used to ensure that the components are not released during the shelf-life of the product but released upon ingestion. Capsule and tablet formulations may be further coated with polymers/materials to prevent extraction of the drug without the inactivating ingredients. Inactivating ingredients, or portions thereof, must be soluble in water and/or alcoholic solutions for the same purpose (to prevent extraction of the drug without the inactivating ingredients).

Other dosage forms or techniques of separation known in the art such as spheronization and drug layering may also be used to improve the stability of the ingredients within the dosage form.

The invention described above should be read in conjunction with the accompanying claims and drawings. The description of embodiments and examples enable one to practice various implementations of the invention and they are not intended to limit the invention to the preferred embodiment, but to serve as a particular example of the invention. Those skilled in the art will appreciate that they may readily use the conception and specific embodiments disclosed as a basis for modifying or designing other methods and systems for carrying out the same purposes of the present invention.

All references, including publications, patent applications, patents, and website content cited herein are hereby incorporated by reference to the same extent as if each reference were individually and specifically indicated to be incorporated by reference and was set forth in its entirety herein.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A drug formulation, comprising:
an active component,
a degrading component capable of degrading the active component,
and a membrane separating the active component from the degrading component,
wherein the membrane prevents degradation of the active component by the degrading component.

2. The drug formulation of claim 1, wherein the membrane is resistant to gastric fluids.

3. The drug formulation of claim 1, wherein the active component is selected from the group consisting of an opiate, an amphetamine, a barbiturate, or a digoxin.

4. The drug formulation of claim 3, wherein the opiate is selected from the group consisting of morphine, codeine, thebaine, papaverine, and derivatives thereof.

5. The drug formulation of claim 3, wherein the amphetramine is selected from the group consisting of methamphetamine [(2S)-N-methyl-1-phenylpropan-2-amine]), MDA (3,4-Methylenedioxyamphetamine), MDMA (3,4-Methylenedioxy-N-methamphetamine), Amphetamine/Benzedrine (1-phenylpropan-2-amine), dextroamphetamine ([(2S)-1-phenylpropan-2-amine]), 4-FMP ([1-(4-fluorophenyl)propan-2-amine]), 4-MTA ([1-[4-(methylthio)phenyl]propan-2-amine], Benzphetamine ([(2S)-N-benzyl-N-methyl-1-phenylpropan-2-amine], and derivatives thereof.

6. The drug formulation of claim 3, wherein the barbiturate is selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), phenobarbital (5-ethyl-5-phenylbarbiturate).

7. The drug formulation of claim 3, wherein the barbiturate is selected from the group consisting of chlordiazepoxide, clorazepate, diazepam, flurazepam, halazepam, prazepam, and others lorazepam, lormetazepam, oxazepam, temazepam, clonazepam, flunitrazepam, nimetazepam, nitrazepam, adinazolam, alprazolam, estazolam, triazolam, climazolam, loprazolam and midazolam

8. The drug formulation of claim 1, wherein the degrading component is selected from the group consisting of an oxidation agent, a hydrolysis agent, or a complexing agent.

9. The drug formulation of claim 8, wherein the oxidation agent is selected from the group consisting of sodium bismuthate, sodium permanganate, potassium permanganate, sodium persulfate, peroxides, and derivatives thereof.

10. The drug formulation of claim 1, wherein the membrane prevents release of the degrading component when the drug formulation is ingested by a patient.

11. The drug formulation of claim 1, wherein the membrane is pH sensitive.

12. The drug formulation of claim 1, further comprising a pH enhancer that is capable of increasing the pH of an individual's stomach when ingested, wherein the pH enhancer is separated from the degrading agent by the membrane.

13. The drug formulation of claim 11, the drug formulation of claim 11, wherein the membrane degrades after the pH of a solution surrounding it reaches a predetermined pH.

14. The drug formulation of claim 13, wherein the membrane degrades when the solution in which it is placed reaches a pH equal or greater than 5.

15. The drug formulation of claim 1, wherein the pH enhancer is selected from the group consisting of an antacid, magnesium oxides, sodium bicarbonate, sodium acetate, and derivatives thereof.

16. The drug formulation of claim 15, wherein the pH enhancer is present in an amount not sufficient to raise the solution's pH above the predetermined pH in a single dose of the drug formulation.

17. The drug formulation of claim 1, further comprising a catalyst (such as ferrous ions or EDTA) that enhances the activity of the degrading component.

18. The drug formulation of claims 1, wherein the membrane is selected from the group consisting of eudragit®, ethylcellulose, silicates, shellac, polysiloxanes, polylactides, polycaprolactones lipids, waxes and other films forming organic and inorganic materials.

19. The drug formulation of claim 7, wherein complexing agent is selected from the group consisting of tanic acid or other complexing agents.
